(19) United States
(12) Patent Application Publication
(10) Pub. No.: US 2026/0220253 A1
(43) Pub. Date: Jul. 30, 2026

(54) ARCHITECTURE FOR SANDBOXING BIOMEDICAL DATA FOR RESEARCH PIPELINES

(71) Applicant: Sequence Bio, St. John's (CA)

(72) Inventors: Alain R. O' Dea, St. John's (CA); Francis A. March, Paradise (CA)

(21) Appl. No.: 19/573,285

(22) Filed: Mar. 20, 2026

Related U.S. Application Data
(63) Continuation of application No. PCT/IB24/00532, filed on Sep. 20, 2024.
(60) Provisional application No. 63/539,932, filed on Sep. 22, 2023.

(51) Int. Cl. G06F 21/53; G16H 10/60

(52) U.S. Cl. CPC G06F 21/53; G16H 10/60; G06F 2221/034

(57) ABSTRACT

A method including accessing a datastore containing therein a plurality of biomedical datasets, generating a plurality of data stores, each containing one of the plurality of datasets, receiving a request to instantiate a research environment, in response to the request, copying at least a subset of at least one of the plurality of datasets to an environment-specific datastore, providing a sandbox comprising one or more application and the environment-specific datastore, invoking the one or more application on the environment-specific datastore, and collecting therefrom a resulting dataset in a results datastore, and providing access to the results datastore to a requester outside the sandbox without providing access to the environment-specific datastore.

### RELATED APPLICATION

This application claims the benefit of U.S. Provisional Application. No. 63/539,932 filed Sep. 22, 2023, which is hereby incorporated by reference in its entirety.

### BACKGROUND

Embodiments of the present disclosure relate to management of complex biomedical datasets, and more specifically, to architectures for sandboxing biomedical data for research pipelines.

### BRIEF SUMMARY

According to embodiments of the present disclosure, methods of and computer program products for architecture for sandboxing biomedical data for research pipelines are provided.

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter provides a method that includes accessing a datastore containing therein a plurality of biomedical datasets. The method can further include generating a plurality of datastores.

Each data store can contain one of the plurality of datasets. In addition, the method can include receiving a request to instantiate a research environment. The method can further include, in response to the request, copying at least a subset of at least one of the plurality of datasets to an environment-specific datastore. The method can further include providing a sandbox including one or more application and the environment-specific datastore. The method can further include invoking the one or more application on the environment-specific datastore. The method can further include collecting therefrom a resulting dataset in a results datastore. The method can then provide access to the results datastore to a requester outside the sandbox. The method can provide access to the results datastore without providing access to the environment-specific datastore.

According to some embodiments of the present disclosure, the method can further include receiving from a remote client an additional dataset and providing the additional dataset in the sandbox.

According to some embodiments of the present disclosure, the plurality of biomedical datasets can include genetic data and/or medical records.

According to some embodiments of the present disclosure, each of the biomedical datasets can have an associated data type. Each of the plurality of datastores can correspond to exactly one of the data types.

According to some embodiments of the present disclosure, each of the plurality of datastores can be an object-based storage.

According to some embodiments of the present disclosure, the request can be provided by a remote client via a network.

According to some embodiments of the present disclosure, the environment-specific datastore can be an object-based storage and/or be organized according to data type.

According to some embodiments of the present disclosure, the sandbox can include a cloud instance.

According to some embodiments of the present disclosure, the invoking can be in response to a request from a remote client via a network.

According to some embodiments of the present disclosure, the results datastore can include an object-based storage.

According to some embodiments of the present disclosure, providing access to the results data store can include applying one or more exfiltration rules.

The disclosed subject matter also includes a system including a computing node comprising a computer readable storage medium having program instructions embodied therewith, the program instructions executable by a processor of the computing node to cause the processor to perform any method as described herein.

The disclosed subject matter also includes a computer program product for sandboxing biomedical data, the computer program product comprising a computer readable storage medium having program instructions embodied therewith, the program instructions executable by a processor to cause the processor to perform any method as described herein.

### BRIEF DESCRIPTION OF THE SEVERAL VIEWS OF THE DRAWINGS

FIG. 1 is an exemplary environment for data analysis in accordance with the disclosed subject matter.

FIG. 2 is an exemplary depiction of a system for research and discovery in accordance with the disclosed subject matter.

FIG. 3 is an exemplary enclave environment in accordance with the disclosed subject matter.

FIG. 4 is a high-level logical architecture of a research and analysis platform in accordance with the disclosed subject matter.

FIG. 5 is a flowchart illustrating a method for sandboxing biomedical data in accordance with the disclosed subject matter.

FIG. 6 depicts a computing node according to embodiments of the present disclosure.

### DETAILED DESCRIPTION

Large sets of genomic data are critical for identification of disease signatures and potential drug targets. However, genomic data are also subject to significant privacy concerns, and their use is often limited by regulations applying to personal medical data and data use agreements between entities. Accordingly, there is a need for a platform that enables controlled access to genomic data for research purposes while limiting exfiltration or misuse.

The present disclosure provides a secure and controlled environment designed to support research activities in a scalable way. In various embodiments, data and analysis capabilities are provided in a modular cloud environment running container-based, cloud-native tools, applications and pipelines. This architecture provides maximum flexibility and function for various analytical needs and is designed with extensibility and future-proofing in mind.

In order to facilitate secure and controlled collaborations, secure cohort enclaves are provided that can share coded data (anonymized and de-identified information) and deploy customized analysis pipelines with collaborators such as academic and commercial partners.

While various embodiments herein focus on genomic data, it will be appreciated that the present disclosure is equally applicable to a variety of biomedical data, including without limitation genomic data, Electronic Health Records (EHRs), sequencing data, and medical imagery.

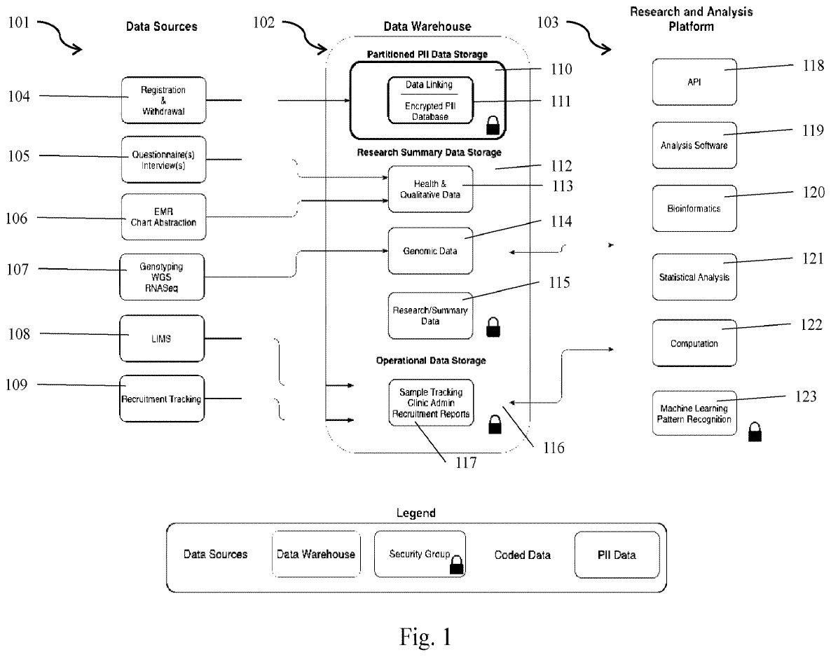

Referring to FIG. 1, an exemplary environment for data analysis is illustrated. Generally, the architecture is divided into three tiers: data sources 101, data warehouse 102, and research and analysis platform 103. The various exemplary data sources 101 are imported into data warehouse 102. Data are stored under specific security groups (lock symbols), each with their own secure and controlled access. All personally identifiable information (PII) is stored in its own partitioned and encrypted database separate from all other data. All research is only performed on coded (de-identified) data within research and analysis platform 103.

In various embodiments, data sources 101 may include registrations and withdrawal information 104, questionnaire(s) and interview(s) 105, EMR and chart abstractions 106, genetic information such as genotyping, WGS, or RNASeq 107, laboratory information management system (LIMS) information 108, recruitment tracking information 109, among other suitable embodiments. In various embodiments, data warehouse 102 may include a partitioned PII data storage 110, providing data linking and encrypted PII database 111. In various embodiments, data warehouse 102 may include research summary data storage 112, including health and qualitative data 113, genomic data 114 and research/summary data 115. In various embodiments, data warehouse 102 may include operational data storage 116 include sampling tracking, clinic admin, and recruitment reports 117. It will be appreciated that each of these data types are exemplary, and additional information may be included in additional embodiments.

Research and analysis platform 103 enables a suite of interconnected applications for health research. In particular, the platform streamlines the complex, compute-intensive tasks of analyzing massive, integrated sets of multi-omic and medical data for research. From custodian-managed data warehouses, full or fractional datasets are mirrored into enclave environments for secure collaboration across internal teams, or with external, domestic and international partners. By bringing researchers to where data resides, data residency and security is enforced by design, and hardened with user access controls, encryption, and data exfiltration prevention configurations.

In various embodiments, research and analysis platform 103 may provide API 118, analysis software 119, bioinformatics 120, statistical analysis 121, computation 122, machine learning (ML) pattern recognition 123, among other suitable components.

In various embodiments, a custodian can instantiate and grant controlled access to collaborative research and analysis enclaves. By default, these online environments are configured with a suite of best-in-class big data analysis, mining, and visualization tools. They can also be pre-populated by custodians with open source or managed datasets from their data warehouse.

Enclaves may be highly configurable to meet the specific needs of a research study's experimental design. Collaborator assets, including proprietary or licensed datasets, computational pipelines, and analysis toolchains, can be safely deployed in enclaves to extend the breadth and depth of its built-in capabilities.

In exemplary embodiments, access to data is managed by custodians through virtual private cloud (VPC) endpoints with custom, narrowly scoped identity and access management (IAM) policies. This ensures that all data is encrypted in transit, remains within the appropriate region-based networks at all times, and that access is restricted to authorized users of an enclave with which the data is shared.

In various embodiments, data exfiltration prevention is provided. For example, a virtual desktop environment may be provided in each enclave with outbound clipboard disabled. Files can be uploaded to but not removed from an enclave. Intentional exports of data can be facilitated following a detailed review and approval on a case by case basis.

In various embodiments, strict control is imposed on Internet access. For example, outbound communications from each enclave can be restricted to domains in that enclave's strict allow list. All other outbound communications may be denied by default.

In various embodiments, enclave-locked credentials are employed. In such cases, API requests made with credentials of any enclave outside that enclave are explicitly denied and logged.

In various embodiments, per-enclave encryption keys are employed. Data transferred to, stored within, and transferred from each enclave is encrypted with data keys wrapped with a distinct master key. This defense-in-depth measure ensures that even if there was a mistake later introduced in permissions, data would not be able to be read by any other enclave.

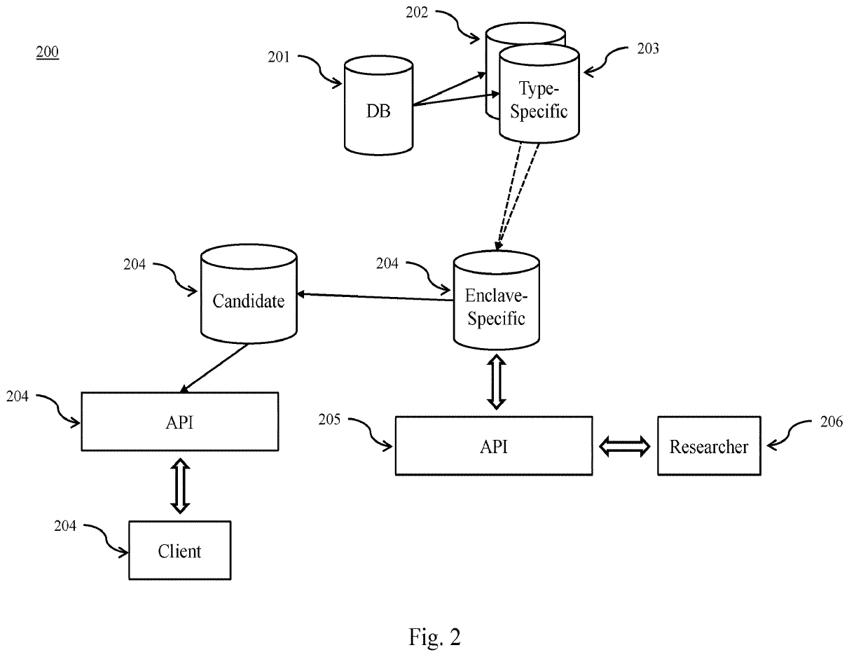

Referring now to FIG. 2, an exemplary depiction of a system 200 for research and discovery is shown in schematic view. In various embodiments, system 200 may include the ability for data to be taken through multiple iterative stages of curation, processing, storage, analysis, integration and/or any other suitable steps towards genetic insights. System 200 may offer a wide set of research and discovery capabilities as shown in FIG. 2. In various embodiments, system 200 may be a secure environment that allows engagement with academic collaborators, commercial partners, and industry partners, among others, to identify novel targets in complex diseases to help address unmet medical need.

Data source 201, which in some embodiments includes data storage 112, includes one or more data types (as exemplified by 113 . . . 115). In order to enable construction of a secure enclave, selected data are first separated out by data type, with each data type being stored in a type-specific data store 202 . . . 203. In some embodiments, each of the type-specific data stores is a cloud-based object storage such as Amazon S3. In various embodiments, each type-specific datastore has an explicit or implicit schema corresponding to its data type.

Upon request for instantiation of a secure enclave, an enclave specific datastore 204 is created. In some embodiments, enclave-specific datastore 204 is a cloud-based object storage such as Amazon S3. In various embodiments, the enclave-specific datastore includes data of multiple types conforming to one or more schema. Providing an enclave specific datastore allows per-enclave permissions and security provisions to be employed. For example, in a cloud environment, there is no need for any access permissions to be granted to the underlying data in type-specific datastores 202 . . . 203 in order to run analytics. In fact access to the underlying datastores may be strictly limited to only. In various embodiments, data are copied from underlying datastores 202 . . . 203 to datastore 204. Read/write access may then be provided to datastore 204, avoiding corruption of the underlying data and avoiding access to data beyond the scope of a given study.

In addition to sandboxing data, the analytics performed on the data are also sandboxed in various embodiments. For example, in some embodiments an API 205 and/or UI is provided for a given researcher 206 to invoke various analytics on the sandboxed data. As discussed below, analytics tools may be preloaded in a sandboxed environment in order to prevent data exfiltration.

In various embodiments, data resulting from the analytics performed on sandboxed data (e.g., summary statistics, individual gene signatures, graphs, etc.) may be loaded into a secondary datastore 206 for review. A candidate release may then be reviewed manually or automatically for sensitive information. For example, a set of exfiltration rules may be applied to the candidate release to detect an attempt at outputting more data than is permitted under relevant handling guidelines. Examples of exfiltration rules include prohibition on PII, prohibition of original sequence data, prohibition on executable code, and limits on overall data size. It will be appreciated that a variety of methods known in the art may be used to detect such prohibited data and flag it as violating one or more exfiltration rules.

In various embodiments, once approved for release, a data access API 206 and/or UI is provided for a client to retrieve and/or view the data approved for release.

As discussed above, clients engaging with the systems described herein for multiple cohorts may be provided with separate enclaves for each cohort in order to ensure the appropriate data access controls and analysis platform for each engagement. Enclaves may be preconfigured with a set of analysis, mining, and visualization tools. In various embodiments, the data are separated in order to make research not schema-specific. In various embodiments, the data types may be recombined in one datastore, organized by folders and directory type. In various embodiments, research may be performed on such a sandbox, then copied into the release datastore.

In various embodiments, cohort data may be entered or pushed into an enclave, then to curated data sets. In various embodiments, client-submitted data may be provided. In various embodiments, analytics tools may be provided in docker images for each tool. In various embodiments, the docker images may be either standard or custom, or a combination of both. In various embodiments, client-supplied and third-party data may be provided.

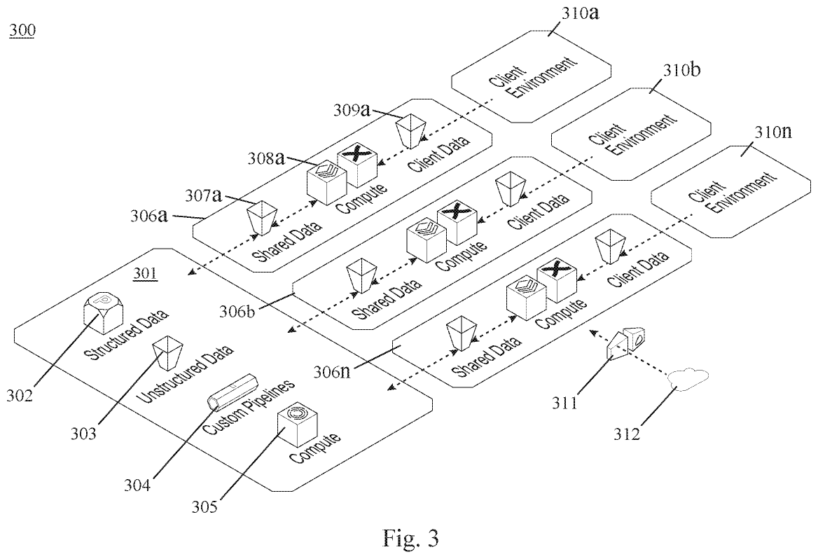

Referring now to FIG. 3, an exemplary enclave environment is shown in schematic diagram view. In various embodiments, the enclave environment 300 provides one or more computational services for research and analysis. As outlined above, cohort enclave system 300 provides a secure environment for data analysis. The security provided includes both protecting the systems' participant's information, and ensuring that the proprietary data of clients utilizing the systems disclosed herein remain safe.

Each enclave may be single-tenanted, thus a collaborator has exclusive access to the enclave. These highly secure, single tenant client environments are created on a per-client and cohort basis. Clients engaging with the systems disclosed herein for multiple cohorts are provided with separate enclaves for each cohort in order to ensure the appropriate data access controls and analysis platform for each engagement. In various embodiments, each cohort enclave environment may be designed for a single tenant, have its own dedicated network and security controls, and only be available to the specific client for which it was commissioned.

Common services 301 may include structured data and unstructured data 303 (e.g., from data warehouse 102). In addition, common services 301 may include various custom analytics pipelines 304 as well as compute 305. Shared data may be loaded into enclave 306a . . . n for analysis within an individual enclave. A given client environment 310a . . . n is assigned a given enclave instance 306a . . . n.

Within each enclave (e.g., 306a), shared data 307a and client data 309a may be combined to perform one or more computation 308a. Resulting data may be read via network 312 (e.g., a public network such as the internet), limited by one or more access restrictions 311.

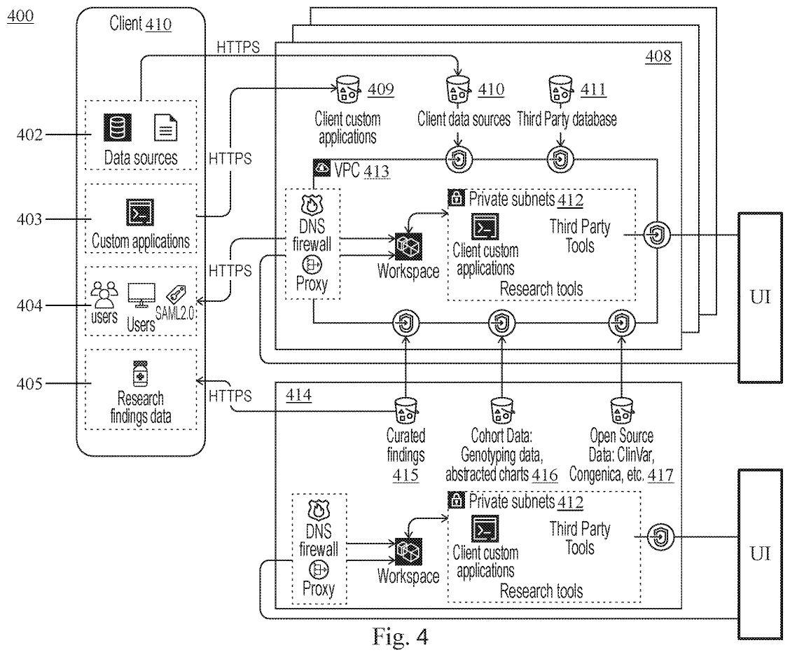

Referring now to FIG. 4, an exemplary high-level architecture of a research and analysis platform in accordance with the disclosed subject matter is provided. As discussed with reference to the prior figures, platform 400 includes one or more enclaves 408. To facilitate secure collaboration with partners and clients, the systems and methods described herein may be implemented in cloud-based environments as exemplified in FIG. 4.

Client 401 may provide one or more data sources 402 and custom applications 403. For hosting in enclave 408 (at 409, 410). These may be combined with one or more third party data sources 411 for further analysis. Client 401 may provide access to one or more users 404. In some embodiments, a virtual private cloud (VPC) is employed for execution of more or more client custom applications and/or third party tools 412.

As discussed in further depth above, cohort data 416 and open source data 417 may be provided to enclave 408 for analysis. Research tools 412 may be run outside the enclave environment, e.g., by a trusted partner within the data management organization. Curated findings 415 may then be provided back to client 401 (at 405).

In various embodiments, any system or method described herein can include or be utilized in cloud services backed by one or more data centers. To ensure data are kept private and confidential, any system described herein may use cloud-based security features that include 24/7 (constant, full time) access monitoring, full disk, regular backups, intrusion detection and file-based encryption, geographically separate redundancy, and advanced physical security of data centers. In various embodiments, advanced physical security of data centers may include video surveillance and 2-factor authentication for entrance into the data center. This technology may also ensure streamlined operations and end-to-end audit trails of system interaction ensuring data security and oversight.

In various embodiments, one or more certified cloud service providers may implement proven security solutions and services for the protection of data, including healthcare and financial data, with clients.

Exemplary embodiments may use Amazon Web Service or equivalent cloud infrastructure providers to implement the systems and architectures set out herein. In various embodiments, to ensure compliance with data management residency requirements, data is maintained on per-country resources. In various embodiments, a cloud platform supports security standards and compliance certification including: HITRUST, GDPR compliance, FedRAMP, HIPAA, ISO 27001, and ISO 3425.

In various embodiments, systems and methods described herein use encryption features to protect its content in transit and at rest, and the systems and methods described herein may manage its own encryption keys. Exemplary embodiments employ AWS Key Management Service (KMS).

The field of genomics is maturing as a demanding domain that requires a complex ecosystem of tools, technologies, computer power and data management capabilities. Software as a Service (SaaS) seeks to overcome challenges associated with software installation and user experience by increasing the degree of automation on software delivery. SaaS is software that is owned, delivered, and managed remotely by a provider who delivers software based on one set of common code and data definitions that is consumed in a one-to-many model by all contracted customers at any time, on a pay-for-use basis or as a subscription. SaaS can address in a convenient manner the three major challenges of science software, namely usability, scalability, and sustainability.

Analysis of high throughput genomics data is a complex and compute-intensive task that generally requires numerous software tools and large reference datasets, tied together in successive stages of data transformation and visualization. In various embodiments, a SaaS workbench for genomics research integrates cloud computing with a suite of bioinformatics tools for analysis and discovery. It may provide a solution for the analysis of massive genomic datasets.

At the heart of research and analysis platforms according to the present disclosure lies the cohort enclave. These highly secure, single tenant born-in-the-cloud client environments are created on a per-client and cohort basis. Each cohort enclave may be built and fully managed as a SaaS service by the systems and methods described herein.

To ensure the most secure environment possible, systems and methods described herein include security in every step of the design and configuration. Data protection was taken into account throughout the entire systems development process. Data exfiltration is the act of deliberately moving sensitive data from inside an organization to outside an organization's perimeter without permission. The systems and methods described herein provide a secure environment that would allow access from external users but prevent unauthorized data exfiltration. This privacy-by-design approach was taken to ensure the most secure enclave environment.

In various embodiments, security measures include firewall-controlled Internet access. There is a strict web filter allow-list in place and access to a site or service may be allowed only if the URL is added to the list. All other web or Internet traffic is denied by default.

In various embodiments, the additional security measures may include data exfiltration prevention. The systems and methods may be configured to not allow files to be copied out of the environment. A strict allow-list of data stores works in tandem with the firewall to allow what is required and deny everything else.

In various embodiments, the additional security measures may include DNS exfiltration prevention. The systems and methods can be configured to implement a DNS firewall with a strict domain allowlist. This prevents the exfiltration of data via DNS queries.

In various embodiments, the additional security measures may include Denial of running IAM policies external to the VPC. The systems and methods can be configured to include extra security precautions to limit the utility of IAM Role credentials outside of the VPC. Attempts to make AWS API requests with the credentials of any enclave IAM Role outside the VPC may be denied.

In various embodiments, the additional security measures may include security group rules. Every security group (both ingress and egress) was thoroughly vetted to ensure that only the most restrictive permissions were allowed.

In various embodiments, the additional security measures may include segregation by account and not just VPC. Rather than just creating a VPC for each potential client in one account, a more secure approach was implemented by giving each client their own account. This allowed a totally segregated environment whose only communication is through S3 buckets. All other operations would be entirely separate.

In various embodiments, the additional security measures may include use of S3 Endpoint and S3 Endpoint policy. Not only are S3 endpoints used to provide a more secure transfer of data to/from S3, (using internal AWS routes and not traversing the internet) but those S3 endpoints are locked down by a policy. Only S3 buckets on the list are allowed to be accessed. This prevents exfiltration by copying to other S3 buckets in AWS.

In various embodiments, the additional security measures may include individual Key Management Services (KMS) keys. Not only does each enclave have its own individual KMS key, but communication with the shared environment is done by a separate key for each enclave. This defense in depth measure ensures that even if there was a mistake later introduced in permissions, data would not be able to be read by any other enclave.

In various embodiments, the additional security measures may include SSO. Login is controlled by SSO backed by a trusted provider such as Google. Only approved email addresses can log in which offers protection from a compromised username/password.

Once the client collaboration is complete and any research findings have been provided to the client, the enclave is cryptographically erased, including all resources contained within. In various embodiments, audit and security logs may be retained beyond the life of the enclave, meeting and exceeding industry best practices.

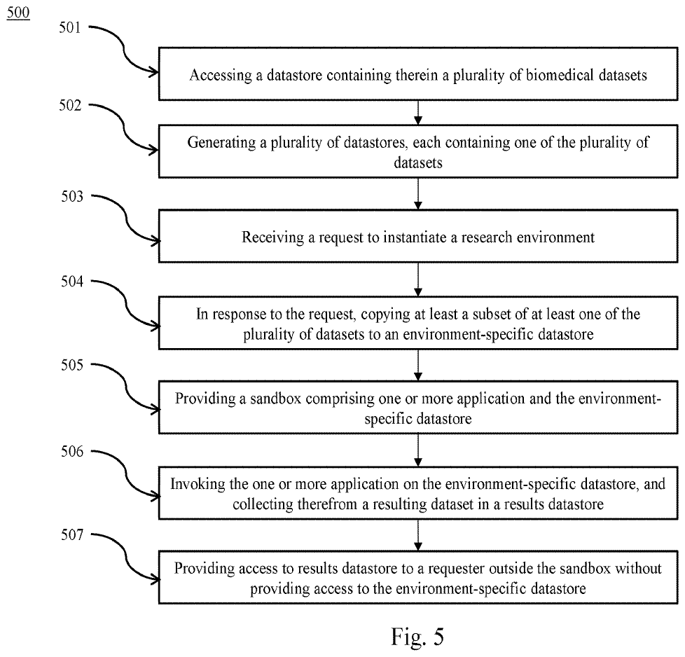

Referring to FIG. 5, a method 500 includes, at step 501, accessing a datastore containing therein a plurality of biomedical datasets. The datastore may be the same or similar to any datastore described herein. The plurality of biomedical datasets may be the same or similar to any biomedical datasets as described herein.

With continued reference to FIG. 5, method 500 includes, at step 502, generating a plurality of datastores, each containing one of the plurality of datasets. The plurality of datastores that are generated by be the same or similar to any datastores as described herein. The plurality of datasets may be the same or similar to any datasets as described herein.

With continued reference to FIG. 5, method 500 includes, at step 503, receiving a request to instantiate a research environment. The research environment may be the same as or similar to any research environment as described herein.

With continued reference to FIG. 5, method 500 includes, at step 504, in response to the request, copying at least a subset of at least one of the plurality of datasets to an environment-specific datastore. The at least a subset of at least one of the plurality of datasets may be similar to or the same as any dataset or subset thereof as described herein. The environment-specific datastore may be similar to or the same as any environment-specific datastore as described herein.

With continued reference to FIG. 5, method 500 includes, at step 505, providing a sandbox including one or more application and the environment-specific datastore. The sandbox may be the same or similar to any sandbox as described herein. The one or more applications may be the same or similar to any applications as described herein. The environment-specific datastore may be similar to or the same as any environment-specific datastore as described herein.

With continued reference to FIG. 5, method 500 includes, at step 506, invoking the one or more application on the environment-specific datastore, and collecting therefrom a resulting dataset in a results datastore. The one or more applications may be the same or similar to any applications as described herein. The environment-specific datastore may be similar to or the same as any environment-specific datastore as described herein. The resulting dataset may be the same or similar to any dataset as described herein. The results datastore may be the same or similar to any results datastore as described herein.

With continued reference to FIG. 5, method 500 includes, at step 507, providing access to results datastore to a requester outside the sandbox without providing access to the environment-specific datastore. The results datastore may be the same or similar to any results datastore as described herein. The requester may be any requester as described herein. The sandbox may be the same or similar to any sandbox as described herein.

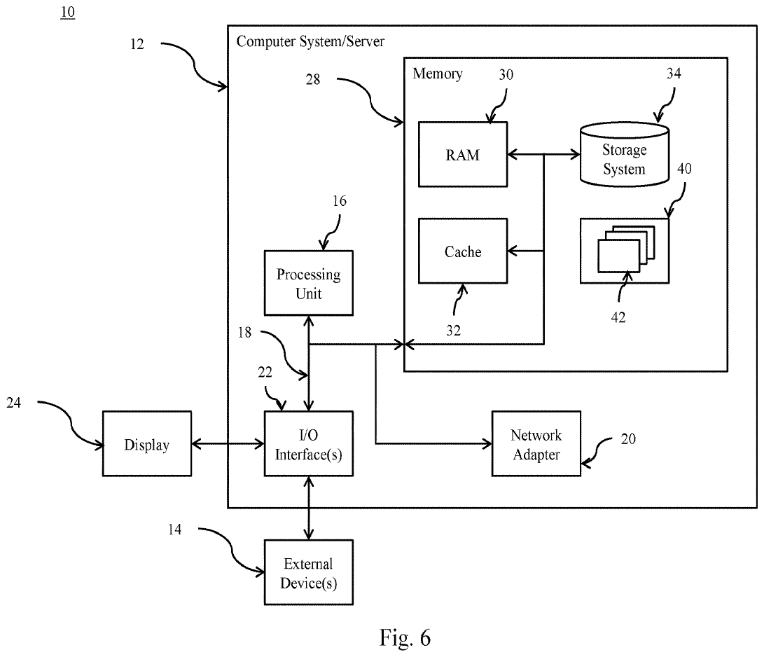

Referring now to FIG. 6, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22.

Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
accessing a datastore containing therein a plurality of biomedical datasets;
generating a plurality of data stores, each containing one of the plurality of datasets;
receiving a request to instantiate a research environment;
in response to the request, copying at least a subset of at least one of the plurality of datasets to an environment-specific datastore;
providing a sandbox comprising one or more application and the environment-specific datastore;
invoking the one or more application on the environment-specific datastore;
collecting therefrom a resulting dataset in a results datastore; and
providing access to the results datastore to a requester outside the sandbox without providing access to the environment-specific datastore.

2. The method of claim 1, wherein the plurality of biomedical datasets comprises genetic data.

3. The method of claim 1, wherein the plurality of biomedical datasets comprises medical records.

4. The method of claim 1, wherein each of the plurality of biomedical datasets has an associated data type, and wherein each of the plurality of datastores corresponds to exactly one data type.

5. The method of claim 1, wherein each of the plurality of datastores is an object-based storage.

6. The method of claim 1, wherein the request is provided by a remote client via a network.

7. The method of claim 1, wherein the environment-specific datastore is an object-based storage.

8. The method of claim 7, wherein the environment-specific datastore is organized according to data type.

9. The method of claim 1, wherein the sandbox comprises a cloud instance.

10. The method of claim 1, wherein said invoking is in response to a request from a remote client via a network.

11. The method of claim 1, wherein the results datastore comprises an object-based storage.

12. The method of claim 1, wherein providing access to the results datastore comprises applying one or more exfiltration rules.

13. The method of claim 1, further comprising:
receiving from a remote client an additional dataset and providing the additional dataset in the sandbox.

14. A system comprising:
a computing node comprising a computer readable storage medium having program instructions embodied therewith, the program instructions executable by a processor of the computing node to cause the processor to perform a method comprising:
accessing a datastore containing therein a plurality of biomedical datasets;
generating a plurality of data stores, each containing one of the plurality of datasets;
receiving a request to instantiate a research environment;
in response to the request, copying at least a subset of at least one of the plurality of datasets to an environment-specific datastore;
providing a sandbox comprising one or more application and the environment-specific datastore;
invoking the one or more application on the environment-specific datastore;
collecting therefrom a resulting dataset in a results datastore; and
providing access to the results datastore to a requester outside the sandbox without providing access to the environment-specific datastore.

15. A computer program product for sandboxing biomedical data, the computer program product comprising a computer readable storage medium having program instructions embodied therewith, the program instructions executable by a processor to cause the processor to perform a method comprising:
accessing a datastore containing therein a plurality of biomedical datasets;
generating a plurality of data stores, each containing one of the plurality of datasets;
receiving a request to instantiate a research environment;
in response to the request, copying at least a subset of at least one of the plurality of datasets to an environment-specific datastore;
providing a sandbox comprising one or more application and the environment-specific datastore;
invoking the one or more application on the environment-specific datastore; collecting therefrom a resulting dataset in a results datastore; and
providing access to the results datastore to a requester outside the sandbox without providing access to the environment-specific datastore.

16. The system of claim 14, wherein the plurality of biomedical datasets comprises genetic data.

17. The system of claim 14, wherein the plurality of biomedical datasets comprises medical records.

18. The system of claim 14, wherein each of the plurality of biomedical datasets has an associated data type, and wherein each of the plurality of datastores corresponds to exactly one data type.

19. The system of claim 14, wherein each of the plurality of datastores is an object-based storage.

20. The system of claim 14, wherein the request is provided by a remote client via a network.
